(19) United States
(12) Patent Application Publication
(10) Pub. No.: US 2025/0359980 A1
(43) Pub. Date: Nov. 27, 2025

(54) Implantable Artificial Bronchus And Use Of An Implantable Artificial Bronchus

(71) Applicant: Pulmair Medical, Inc., San Diego, CA (US)

(72) Inventor: Murilo Pundek Rocha, São Paulo (BR)

(73) Assignee: Pulmair Medical, Inc., San Diego, CA (US)

(21) Appl. No.: 19/293,359

(22) Filed: Aug. 7, 2025

Related U.S. Application Data
(63) Continuation of application No. 17/381,634, filed on Jul. 21, 2021, now Pat. No. 12,402,998.
Application No. 17/381,634 is a continuation of application No. 16/055,686, filed on Aug. 6, 2018, now Pat. No. 11,096,773.
Application No. 16/055,686 is a continuation of application No. 15/156,841, filed on May 17, 2016, now Pat. No. 10,806,560.

(30) Foreign Application Priority Data
May 18, 2015 (BR) 10 2015 0113765

(51) Int. Cl. A61F 2/04; A61F 2/24

(52) U.S. Cl. CPC A61F 2/04; A61F 2/2476; A61F 2002/043; A61F 2210/0071; A61F 2230/0067; A61F 2230/0069; A61F 2250/0069; A61F 2310/00023

(57) ABSTRACT

An implantable artificial bronchus (IAB) is provided that is used for the treatment of chronic obstructive pulmonary diseases, such as pulmonary emphysema. The implantable artificial bronchus can be made with silicone or nitinol, and has a tapered cylindrical shape. Additional embodiments of this apparatus may be further associated with a one-way valve on the nozzle of the IAB.

### FIELD OF THE INVENTION

The instant patent of invention relates to artificial bronchi for the treatment of pulmonary emphysema, and may be associated with a one-way valve.

### BACKGROUND OF THE INVENTION

Pulmonary emphysema is a type of chronic obstructive pulmonary disease (COPD), which is characterized by the permanent enlargement of the gas exchange units (acini) associated with the destruction of alveolar walls, without fibrosis.

The destruction of these alveolar walls shows distinct patterns in each person, with different distribution and intensity. This gradual and irreversible rupture of lung tissue leads to the loss of the elastic capacity of lung recoil, namely, the loss of the ability to expel inspired air. As a result, there is a structural derangement of the rib cage, increase of the thorax diameters and diaphragm rectification (air entrapment and hyperinflation)

Areas affected by the disease fill the thoracic cavity, leaving less volume available for the healthy areas of lung tissue perform hematosis. The loss of respiratory capacity leads to a progressive functional disability of individuals with the disease.

Although there is no cure for pulmonary emphysema, there are some forms of treatment, including medicines, muscle training and oxygen therapy. For some more severe cases, a lung volume reduction surgery or lung transplantation may be indicated. While the medicines and muscle training have limited results, the lung volume reduction surgery and lung transplantation are very traumatic, and a very limited number of patients can be submitted to these treatments.

Since the year 2000, several less traumatic alternatives have been proposed to alleviate the suffering of patients with pulmonary emphysema.
a) Methods based on forced scarring of the sick tissue using chemicals or steam to produce mechanical retraction. These techniques are still being assessed and have the risk of producing or accelerating the damage caused by the disease itself after an initial improvement.
b) The European patent EP1524942 describes a self-expanding nitinol endobronchial valve covered by a silicone membrane, which has a structure that allows unidirectional air flow. This valve is implanted in areas close to the affected regions, causing the air accumulated therein to be expelled. Due to its unidirectional characteristic, it does not allow the reentry of air into the affected areas. The problem of this technique is the collateral communication between the entrapped air in the treated area and the remaining parts of the same lung. Another problem is that the working tissue should be excluded, for there to be a beneficial effect.
c) An alternative approach was the creation of ancillary air passages in the bronchi wall, to allow lung emptying. Although this alternative presents a more efficient solution over the affected lung, the existing state of the art results demonstrated the premature closure of these passages.
d) There is also treatment with mechanical retraction of the airways, where nitinol spirals (nickel-titanium alloy) are implanted using a straightened bronchoscope and, after the implant, the covers that keep the spiral straightened are removed, causing it to return to its original conformation, thus performing the retraction of the pulmonary parenchyma. This method is limited due to its irreversibility, inability of using an anticoagulant medication (for circulatory illnesses), a very high risk in patients with high pulmonary arterial pressure and lack of effect on patients affected with an advanced form of the disease, when there is not almost lung parenchyma.

Thus, there is the need for an alternative, a procedure that is not aggressive to the lung and that promotes lung disinsufflation without excluding its healthy areas.

### BRIEF DESCRIPTION OF THE FIGURES

FIGS. 1 and 2 respectively represent the front and side views of an embodiment of the implantable artificial bronchus (1), according to this invention.

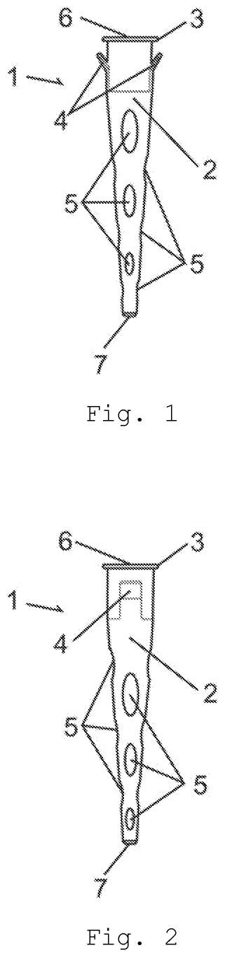

FIGS. 3 and 4 respectively represent upper and lower profile views of the implantable artificial bronchus (1).

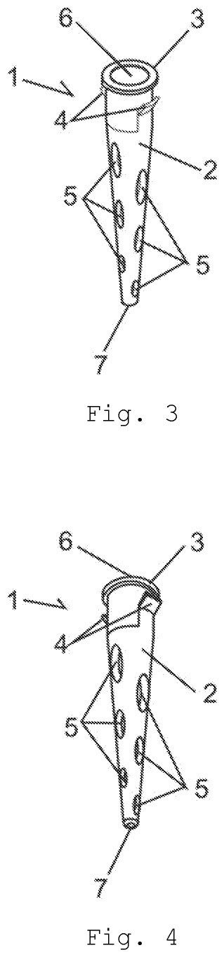

FIG. 5 shows the upper view of the implantable artificial bronchus (1).

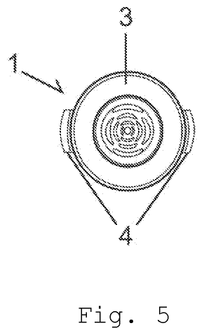

FIG. 6 represents a lung showing the branches of the airway. The arrows represent compression that the air exerted on the same.

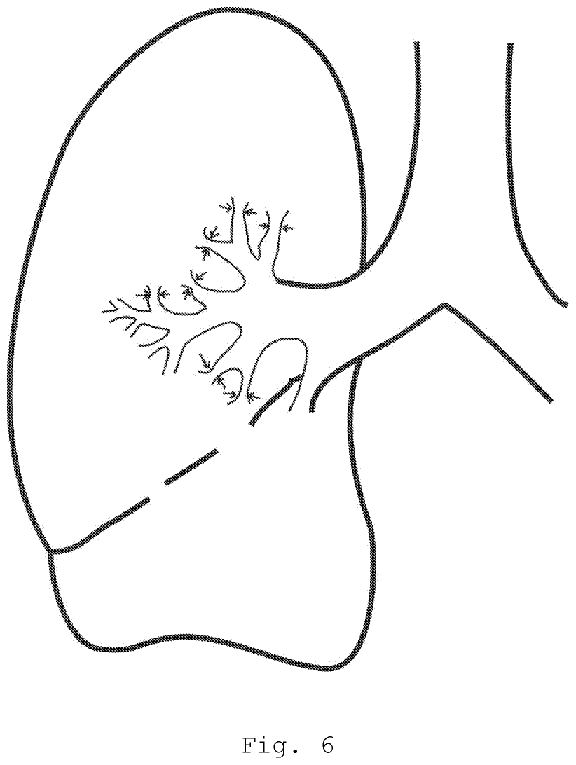

FIG. 7 shows the use of an implanted embodiment of implantable artificial bronchus, according to the present invention.

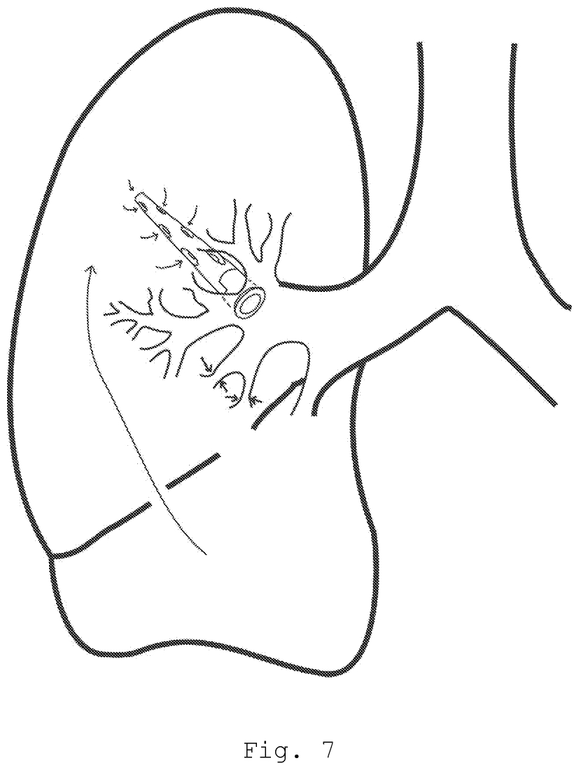

FIG. 8 represents another embodiment of the implantable artificial bronchus, made with a nitinol web.

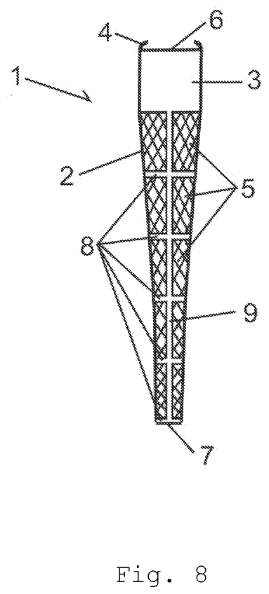

FIG. 9 represents the use of another embodiment of the implantable artificial bronchus.

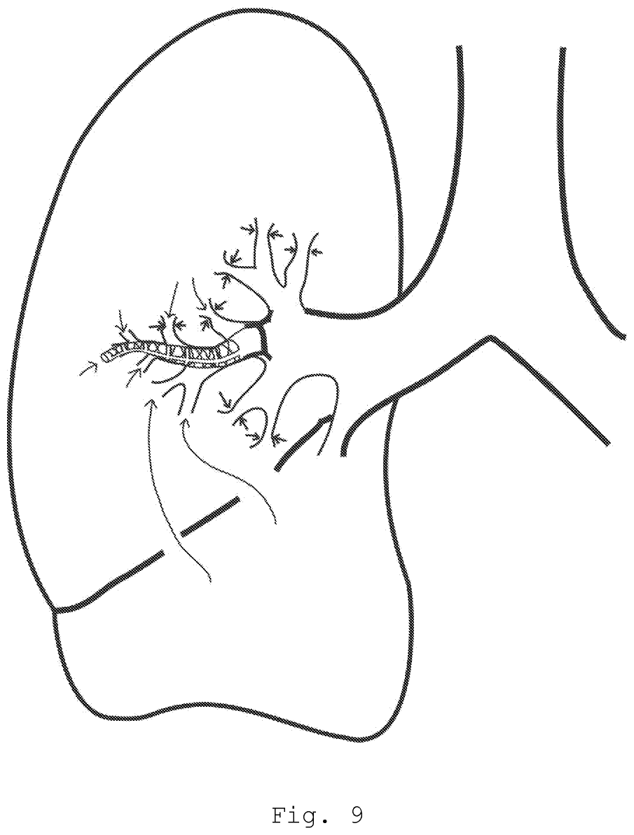

### DESCRIPTION OF THE INVENTION

In order to meet the need for an effective form of treatment, which is not an aggressive or an invasive procedure for the body, an implantable artificial invasive bronchus (IAB) was developed.

The IAB, according to the present invention, consists of a tapered cylindrical body, where the upper nozzle is greater than the lower opening, comprising openings along its length and side wings.

In this invention, openings are any kind of side perforation or leakage from a network assembly.

The IAB (1) can be built in two alternative forms, with a silicone body (SB) or a nitinol web body (NWB), wherein both comprise in the body (2) a nozzle (3) and side wings (4), with side openings (5), upper (6) and lower (7) which enable the peripheral range for promoting lung deflation, which may have various longitudinal lengths.

In the SB assembly, the side wings are used for fixing the same in the air way/lung parenchyma, and in the NWB assembly, they enable a better handling, in case of IAB withdrawal.

The NWB embodiment further has silicone or fluoropolymer like polytetrafluoroethylene rings (8) and longitudinal rods (9), of silicone or fluoropolymer like polytetrafluoroethylene and these structures prevent the incorporation of the nitinol web by the lung wall, and also preserves the possibility of IAB withdrawal in case of rejection or need to reimplant.

Another embodiment of this invention is the association of IAB with a one-way valve, as described in EP1524942—of H. Michael to Emphasys Medical Inc., published on Apr. 27, 2005, incorporated herein by reference. This association prevents lung parenchyma against injuries caused by dry air and moisture reduction, thus avoiding tissue reaction and closure by healing of those openings.

The IAB (1) features a (proximal) upper opening (6) which allows the association with the one-way valve, and also its maintenance, since it allows the removal of the valve, device cleaning with broncoscope and valve reimplantation.

The use of this device is made by bronchoscopy. Initially, it is necessary to identify the locations where it is desired to carry out the application, through an image study obtained by computed tomography of the thorax, associated or not to a three-dimensional reconstruction program.

After identifying the positions, the application can be performed in two ways, one for IAB with SB, which begins with the passage of the needle for piercing the bronchial wall and introducing the guide wire in the lung parenchyma, and optionally, the balloon dilator can be passed.

The implanting of IAB with NWB does not require perforations into the lung wall. The implant path is initially identified with a malleable metal guide. A subsequent catheter passage can be done to guide the compressed IAB or the compressed IAB can be introduced directly by guidewire. After withdrawing the catheter, the IAB naturally expands and remain on the airway, promoting the enlargement of this path and providing causing lung deflation.

The present invention avoids the state of the art problems, since its cylindrical body allows the implantation without the need for extensive cuts or openings that trigger healing processes, or along the airways. Moreover, in the second embodiment according to the present invention, there is no need for any perforation except eventually for very distal airway. Additionally, its conformation with decreasing radius along the body, with side openings, promotes swirling of the air which enters the IAB. Thus, there is no sudden entry and the air is dispersed more evenly, thus ensuring an efficient and safe distribution, without causing tissue healing.

The use of IAB provides more safety and effectiveness in the treatment of lung emphysema, since it allows the air exit, does not trigger the healing mechanisms and does not destroy or annul the normal lung tissue.

It shall be understood that the embodiments described above are merely illustrative and any modification to them may occur for a person skilled in the art. Therefore, the present invention should not be considered as being limited to the embodiments described in this document.

The person skilled in the art will be able to readily evaluate, by means of the teachings contained in the text and in the presented examples, advantages of the invention, and to propose modifications and equivalent alternatives to the embodiments, without departing from the scope of the invention, as defined in the attached claims.

1. An implantable artificial bronchus, comprising:
a tapered cylindrical body;
a nozzle;
side wings;
side openings, and
upper and lower openings.

2. The implantable artificial bronchus, according to claim 1, wherein the body is made with silicone, and include the side wings for attachment, and wherein the side openings are perforations along the body.

3. The implantable artificial bronchus, according to claim 1, wherein the body is made with nitinol web, and include wings for better handling, and wherein the openings consist of leaks from the network assembly.

4. The implantable artificial bronchus, according to claim 3, further comprising silicone or fluoropolymer like polytetrafluoroethylene rings and silicon or fluoropolymer like polytetrafluoroethylene rod.

5. The implantable artificial bronchus, according to claim 1, wherein the implantable artificial bronchus is associated with a one-way valve.

6. The implantable artificial bronchus, according to claim 1 for treating chronic obstructive pulmonary diseases.
